(19) United States
(12) Patent Application Publication
(10) Pub. No.: US 2026/0297123 A1
(43) Pub. Date: Oct. 1, 2026

(54) METHODS FOR THE SYNTHESIS OF NUCLEOSIDE ANALOGUES AND NUCLEOSIDES ANALOGUES DERIVED THEREFROM

(71) Applicant: SIMON FRASER UNIVERSITY, Burnaby (CA)

(72) Inventors: Robert BRITTON, North Vancouver (CA); Garrett MUIR, Halifax (CA); Matthew James ANKETELL, Burnaby (CA); Cohan HUXLEY, Burnaby (CA); Suping BAIKABI, Vancouver (CA)

(73) Assignee: SIMON FRASER UNIVERSITY, Burnaby, BC (CA)

(21) Appl. No.: 19/469,118

(22) PCT Filed: Mar. 28, 2024
(86) PCT No.: PCT/IB2024/053065

§ 371 Date: Sep. 25, 2025

Related U.S. Application Data
(60) Provisional application No. 63/492,967, filed on Mar. 29, 2023.

(51) Int. Cl. C07H 19/067; A61K 31/7072; A61K 31/7105

(52) U.S. Cl. CPC C07H 19/067; A61K 31/7072; A61K 31/7105

(57) ABSTRACT

The present invention relates to methods for the synthesis of nucleoside analogues. More specifically, the present invention relates to methods for the synthesis of C4′ substituted nucleoside analogues via the reaction of a soft nucleophile with a halohydrin ketone.

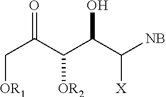

### FIELD

The present invention relates to methods for the synthesis of nucleoside analogues. More specifically, the present invention relates to methods for the synthesis of C4′ substituted nucleoside analogues.

### BACKGROUND

Nucleosides play key roles in diverse cellular processes ranging from cell signalling to metabolism (1). Nucleosides are composed of a nucleobase—canonically composed of adenine, guanine, cytosine, thymine and uracil, and a sugar moiety, typically ribose of 2′-deoxyribose. Nucleosides can be further modified with a 5′-phosphate or phosphate-like group, and RNA oligomers include nucleotides linked via phosphate or phosphate-like linkages from 5′ to 3′. Nucleosides can be modified in several ways, including modifications to the ribose moiety, modifications to the base moiety or modifications to the phosphate moiety, leading to compounds referred to as “nucleoside analogues” (NAs).

NAs have a long and rich history in the field of medicinal chemistry and as tool compounds in chemical biology. The naturally occurring nucleosides are a unique and valuable starting point for drug design due to their involvement in numerous biological processes. Synthetic NAs have been designed to mimic their natural counterparts (2-18). Single NAs have been primarily used as treatments for parasitic, bacterial and fungal infections as well as potent and effective anticancer drugs. In addition to this “small molecule” modality, NAs can be incorporated into oligomeric structures that can modulate gene expression, thus bypassing the complexities associated with protein inhibition. Such oligomeric structures can include short interfering RNA (siRNA), microRNA (miRNA), inhibitory antisense oligonucleotides (ASOs), small activating RNA (saRNA) and messenger RNA (mRNA).

NAs have been used in the treatment of cancer (2, 6) and represent the largest class of small molecule antivirals (3, 4). Mechanistically, NAs can operate as toxic antimetabolites that interfere with nucleic acid synthesis (4). Alternatively, following in vivo phosphorylation, the resulting nucleotide analogues can inhibit enzymes involved in cancer cell growth or viral replication (e.g., DNA/RNA polymerases, ribonucleotide reductases or nucleoside phosphorylases) (2, 4). NAs have also demonstrated promise as epigenetic modulators, and both decitabine and azacitidine inhibit DNA methyltransferase and have been approved for cancer therapy (4).

While several decades of organic and medicinal chemistry have yielded numerous valuable nucleoside analogues, the synthesis of further nucleoside analogues presents some challenges. Nucleoside analogues are often synthesized from naturally occurring carbohydrate, which limits patterns of substitution and furanose stereochemistry (e.g., 19-29). The addition of nucleobases to activated ribose derivatives often fails or proceeds with poor diastereoselectivity with C2′ or C4′ modified nucleosides and efficient strategies for producing C4′ modified nucleosides, including thionucleosides are limited. Synthesis of nucleoside and nucleosides analogues have been described in Meanwell et aL., Science, 369, 725-730 (2020) and WO 2021/191830.

### SUMMARY

The present invention relates to methods and intermediates for the synthesis of C4′ substituted nucleoside analogues.

In one aspect, there is provided a method of synthesizing a C4′ substituted nucleoside analogue, by providing a halohydrin ketone compound of the structure:

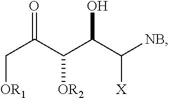

where X may be a halogen; NB may be optionally substituted aryl, arylalkyl, heteroaryl, or heteroarylalkyl; and R₁ and R₂ may each independently be H, alkyl, ester, or protecting group or may together be cyclic; and reacting the halohydrin ketone compound with a soft nucleophile to yield a C4′ substituted nucleoside analogue of the structure:

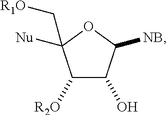

where NB may be optionally substituted aryl, arylalkyl, heteroaryl, or heteroarylalkyl; Nu may be a soft nucleophile; R₁ may be H, alkyl, ester, protecting group, or a covalent bond to Nu, and R₂ may be H, alkyl, ester, protecting group, or a covalent bond to Nu.

In alternative embodiments, R₁ and R₂ may each independently be —OH, —OC(CH₃)₂O—, —(CH₂)₃—, —CH₂SCH₂—, or —CH₂OCH₂—.

In alternative embodiments, the halohydrin ketone compound may be any one of

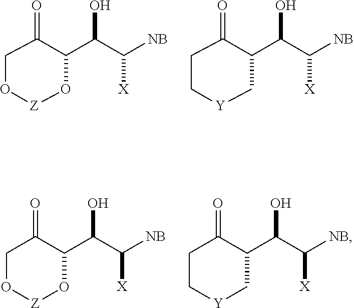

where X may be a halogen; NB may be optionally substituted aryl, arylalkyl, heteroaryl, or heteroarylalkyl; Y may be CH₂, O, S, NR, where R may be alkyl, aryl, acyl, or carboxylate; Z may be a protecting group for an alcohol. In some embodiments, R may be tert-butyloxycarbonyl, benzyloxycarbonyl, or propargyl.

In alternative embodiments, the halohydrin ketone compound may be either of

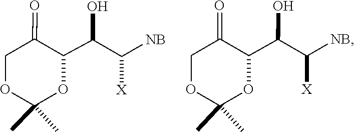

where NB may be optionally substituted aryl, arylalkyl, heteroaryl, or heteroarylalkyl and X may be a halogen.

In alternative embodiments, the halohydrin ketone compound may be either of

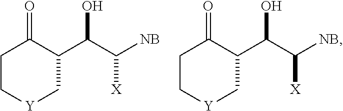

where NB may be optionally substituted aryl, arylalkyl, heteroaryl, or heteroarylalkyl, X may be a halogen, and Y may be CH₂, O, S, NR, where R may be alkyl, aryl, acyl, or carboxylate.

In alternative embodiments, the halohydrin ketone compound may be

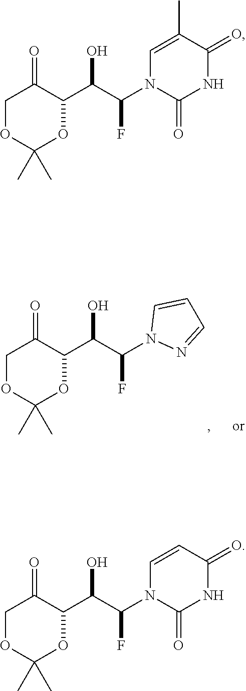

In alternative embodiments, the method of synthesizing a C4′ substituted nucleoside analogue, includes: providing a halohydrin ketone compound of the structure

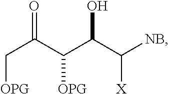

where X may be a halogen; NB may be optionally substituted aryl, arylalkyl, heteroaryl, or heteroarylalkyl; and PG may be a protecting group; and reacting the halohydrin ketone compound with a soft nucleophile to yield a C4′ substituted nucleoside analogue of the structure:

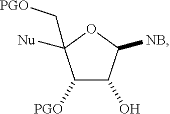

where NB may be optionally substituted aryl, arylalkyl, heteroaryl, or heteroarylalkyl; Nu may be a soft nucleophile; PG may be a protecting group that can optionally form a covalent bond to Nu.

In alternative embodiments, the protecting group for an alcohol may be acetonide, silyl protecting group, alkyl protecting group or aryl protecting group.

In alternative embodiments, the protecting group for an alcohol may be tert-butyl(dimethyl)sily/(TBS), triisopropylsilyl (TIPS), trimethylsilyl (TMS), tert-butyldiphenylsilyl (TBDPS), triethylsilyl (TES), 2-(trimethylsilyl)ethoxymethyl ether (SEM), tetrahydropyranyl (THP) ether, acetate (Ac), pivalate (Piv), methoxymethyl or p-methoxybenzyl (PMB).

In alternative embodiments, the soft nucleophile may be MeCN, N₃⁻, CN⁻, or MeNO₂.

In alternative embodiments, the halogen may be F.

In alternative embodiments, NB may be phenyl, biphenyl, naphthyl, indanyl, indenyl, tetrahydronaphthyl, 2,3-dihydrobenzofuranyl, dihydrobenzopyranyl, 1,4-benzodioxanyl, furan, thiophene, pyrrole, oxazole, thiazole, imidazole, pyrazole, isoxazole, isothiazole, 1,2,3-oxadiazole, triazole, 1,3,4-thiadiazole, tetrazole, pyridine, pyridazine, pyrimidine, 2,6-dichloropyrimidine pyrazine, 1,3,5-triazine, imidazole, benzimidazole, benzoxazole, benzothiazole, indolizine, indole, isoindole, benzofuran, benzothiophene, 1 H-indazole, purine, 4H-quinolizine, quinoline, isoquinoline, cinnoline, phthalazine, quinazoline, quinoxaline, 1,8-naphthyridine, pteridine, uracil, thymine, deazadenine, phthalimide, adenine, 5,6-dihydrouracil, 5-methylcytosine, 5-hydroxymethylcytosine, 5,5,5-trifluoromethylthymine, 5-fluorouracil, 2-thiouracil, 4-methylbenzimidazole, hypoxanthine, 7-deazaguanine, or 7-deazaadenine.

In alternative embodiments, the nucleoside analogue may be a locked nucleic acid (LNA), a small molecule therapeutic, or a monomer in an oligonucleotide therapeutic.

In alternative embodiments, the nucleoside analogue may include a nitrile moiety can be reduced to an aldehyde.

In alternative embodiments, the C4′ substituted nucleoside analogue may be:

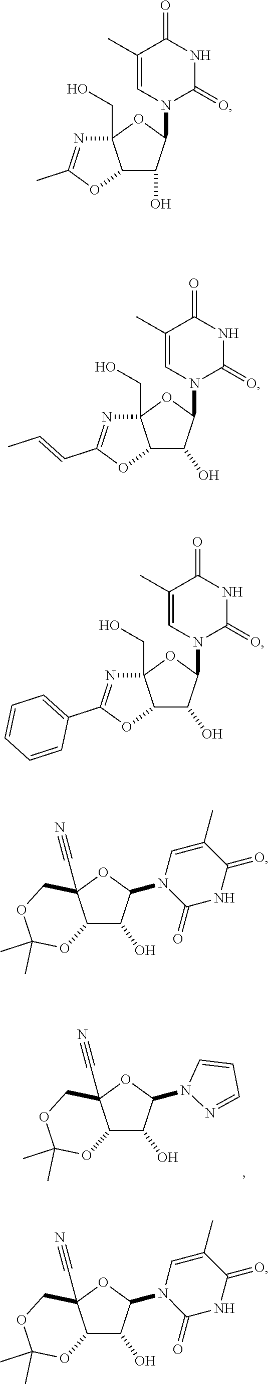

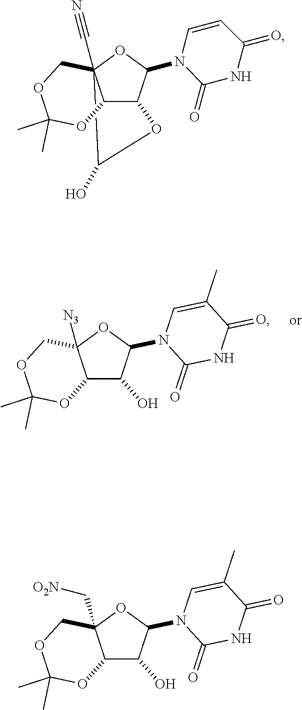

In an alternative aspect, there is provided a C4′ substituted nucleoside analogue:

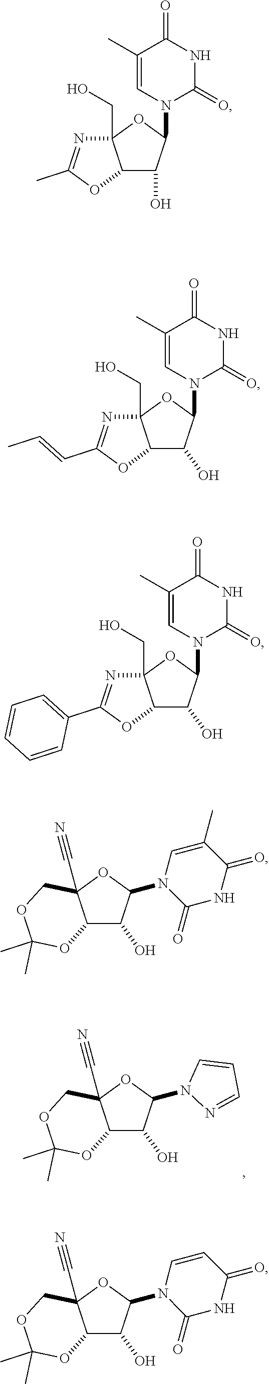

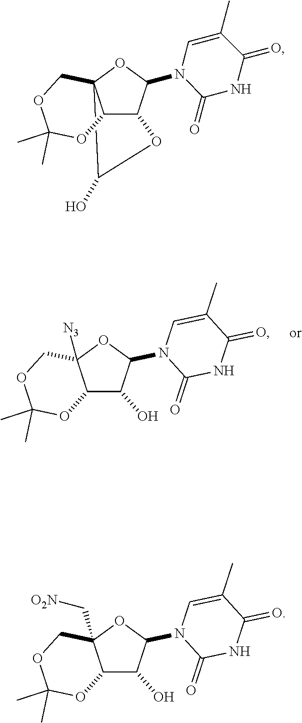

This summary of the invention does not necessarily describe all features of the invention.

### DETAILED DESCRIPTION

The present disclosure provides, in part, methods for the synthesis of nucleoside analogues, such as C4′ substituted nucleoside analogues (NAs).

In some embodiments, the present disclosure provides a method of synthesizing a C4′ substituted NA by reducing a halohydrin ketone compound with a soft nucleophile.

In some embodiments, the present disclosure provides a method of synthesizing a C4′ substituted NA by reacting a halohydrin ketone compound of the structure:

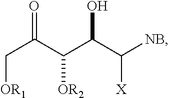

where X may be a halogen; NB may be optionally substituted aryl, arylalkyl, heteroaryl, or heteroarylalkyl; and R₁ and R₂ may each independently be H, alkyl, ester, or protecting group or may together be cyclic; and reacting the halohydrin ketone compound with a soft nucleophile to yield a C4′ substituted nucleoside analogue of the structure:

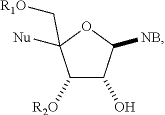

where NB may be optionally substituted aryl, arylalkyl, heteroaryl, or heteroarylalkyl; Nu may be a soft nucleophile; R₁ may be H, alkyl, ester, protecting group, or a covalent bond to Nu, and R₂ may be H, alkyl, ester, protecting group, or a covalent bond to Nu.

A nucleophile is an atom or molecule that contains an electron pair available for bonding with a nucleus of another molecule, to form a covalent bond, for example, a neutral molecule with a lone pair, or a deprotonated molecule. pKa may be correlated to both nucleophilicity and leaving group ability. The pKa values of many common species can be found, for example, as listed below (from http://ccc.chem.pitt.edu/wipf/MechOMs/evans_pKa_table.pdf):

| | | | |
|---|---|---|---|
| Substrate | pka | H₂O | (DMSO) |

INORGANIC ACIDS

| | | |
|---|---|---|
| H₂O | 15.7 | (32) |
| H₃O⁺ | −1.7 | |
| H [symbol] S | 7.00 | |
| HBt | [symbol] .00 | (0.9) |
| HCl | −8.0 | (1.6) |
| HF | 3.17 | (15) |
| HOCl | 7.5 | |
| HClO₄ | −10 | |
| HCN | 9.4 | (12.9) |
| HN₃ | 4.72 | (7.9) |
| HSCN | 4.00 | |
| H₂SO₃ | 1.9, 7.21 | |
| H₂SO₄ | −3.0, 1.99 | |
| H₃PO₄ | 2.12, 7.21, 12.32 | |
| HNO₃ | −1.3 | |
| HNO₂ | 3.29 | |
| H₂CrO₄ | −0.98, 6.50 | |
| CH₃SO₃H | −2.6 | (1.6) |
| CF₃SO₃H | −14 | (0.3) |
| NH₄Cl | 9.24 | |
| B(OH)₃ | 9.23 | |
| HOOH | 11.6 | |

CARBOXYLIC ACIDS

| | | |
|---|---|---|
| 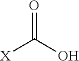 | | |
| X = CH₃ | 4.76 | (12.3) |
| CH₂NO₂ | 1.68 | |
| CH₂F | 2.66 | |
| CH₂Cl | 2.86 | |
| CH₂Br | 2.86 | |
| CH₂I | 3.12 | |
| CHCl₂ | 1.29 | |
| CCl₃ | 0.65 | |
| CF₃ | −0.25 | |
| H | 3.77 | |
| HO | 3.6, 10.3 | |
| C₆H₅ | 4.2 | (11.1) |
| [symbol] -O₂NC₆H₄ | 2.17 | |
| m-O₂NC₆H₄ | 2.45 | |
| p-O₂NC₆H₄ | 3.44 | |
| [symbol] -ClC₆H₄ | 2.94 | |
| m-ClC₆H₄ | 3.83 | |
| p-ClC₆H₄ | 3.99 | |
| o-(CH₃)₃N⁺C₆H₄ | 1.37 | |
| p-(CH₃)₃N⁺C₆H₄ | 3.43 | |
| p-OMeC₆H₄ | 4.47 | |
| 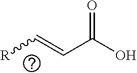 | | |
| R = H | 4.25 | |
| trans-CO₂H | 3.02, 4.38 | |
| cis-CO₂H | 1.92, 6.23 | |

ALCOHOLS

| | | |
|---|---|---|
| HOH | 15.7 | (31.2) |
| MeOH | 15.54 | (27.9) |
| i-PrOH | 16.5 | (29.3) |
| t-BuOH | 17 | (29.4) |
| c-hex₃COH | 24 | |
| CF₃CH₂OH | 12.5 | (23. [symbol] ) |
| (CF₃)₂CHOH | | (17.9) |
| C [symbol] H₅OH | 8.95 | (18.0) |
| m-C₂NC₆H₄OH | 8.35 | |
| p-O₂NC₆H₄OH | 7.14 | (10.8) |
| p-OMeC₆H₄OH | 10.20 | (19.1) |
| 2-napthol | | (17.1) |

OXIMES & HYDROXAMIC ACIDS

| | | |
|---|---|---|
| 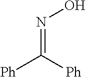 | 11.3 | (20.1) |
| 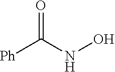 | 8.88 (NH) | (13.7) |
| 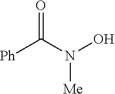 | | (16.5) |

PEROXIDES

| | |
|---|---|
| MeOOH | 11.5 |
| CH₃CO₃H | 8.2 |

PROTONATED SPECIES

| | |
|---|---|
| 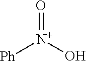 | −12.4 |
| 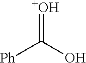 | −7.8 |
| 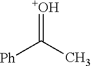 | −6.2 |
| 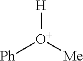 | −6.5 |
| 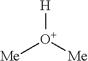 | −3. [symbol] |
| 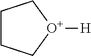 | −2.05 |
| 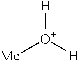 | −2.2 |
| 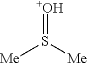 | 4.8 |
| 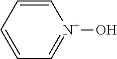 | [symbol] .79 |

SULFINIC & SULFONIC ACIDS

| | |
|---|---|
| 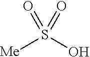 | −2.6 |
| 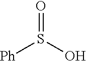 | [symbol] .1 |

*Values <0 for H₂O and DMSO, and values >14 for water and >35 for DMSO were
extrapolated using various methods. [symbol]
indicates data missing or illegible when filed

| | | | |
|---|---|---|---|
| Substrate | pKa | H₂O | (DMSO) |

PROTONATED NITROGEN

| | | | |
|---|---|---|---|
| N⁺H₄ | | 9.2 | (10.5) |
| EtN⁺H₃ | | 10.6 | |
| i-Pr₂N⁺H₂ | | 11.05 | |
| Et₃N⁺H | | 10.75 | (9.00) |
| PhN⁺H₃ | | 4.6 | (3.6) |
| PhN⁺(Me)₂H | | 5.20 | (2.50) |
| Ph₂N⁺H₂ | | 0.78 | |
| 2-naphthal-N⁺H₃ | | 4.16 | |
| H₂NN⁺H | | 8.12 | |
| HON⁺H₃ | | [symbol] .96 | |
| Quinuclidine | 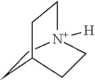 | 11.0 | (9.80) |
| Morpholine | 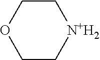 | 8.36 | |
| N-Me morpholine | | 7.38 | |
| 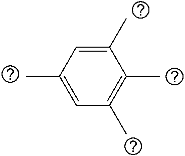 | | −9.3 | |
| DABCO | 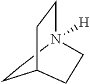 | 2.97, 8.82 (2.97, 8.93) | |
| 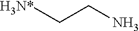 | | 6.90, 9.95 | |
| Proton Spong [symbol] | 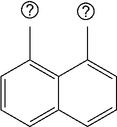 | −9.0, 12.0 ( [symbol] , 7.50) | |
| PhCN⁺H | | −10 | |

AMINES

| | | | |
|---|---|---|---|
| HN₃ | | 4.7 | (7.9) |
| NH₃ | | 38 | (41) |
| i-Pr₂NH | (36 THF)) | | |
| TMS₂NH | 26 (THF) | | (30) |
| PhNH₂ | | | (30.5) |
| Ph₂NH | | (25.0) | |
| NCNH₂ | | | (16.9) |
| 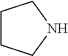 | | | (44) |
| TMP | 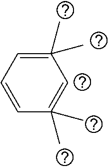 | | (37) |
| 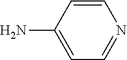 | | | (26.5) |

AMIDES & CARBAMATES

| | | | |
|---|---|---|---|
| 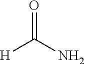 | | | |
| R = H | | | (23.5) |
| CH₃ | | 15.1 | (25.5) |
| Ph | | | (23.3) |
| CF₃ | | | (17.2) |
| NH₂ (urea) | | | (26.9) |
| OEt | | | (24.6) |
| 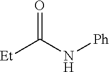 | | | (21.6) |
| 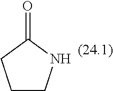 | 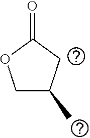 | 12 | (20.5) |

IMIDES

| | | | |
|---|---|---|---|
| 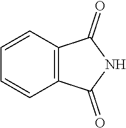 | 6.30 | 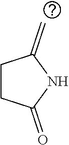 | (14.7) |
| Ac₂NH | | | (17.9) |

SULFONAMIDE

| | | |
|---|---|---|
| MeSO₂NH₂ | | (17.5) |
| PhSO₂NH₂ | | (16.1) |
| CF₃SO₂NH₂ | 6.3 | (9.7) |
| MeSO₂NHPh | | (12.9) |

GUANIDINIUM,
HYDRAZONES, -IDES & -INES

| | |
|---|---|
| 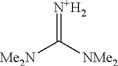 | (13.5) |
| 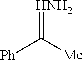 | (21.6) |
| 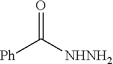 | (18.9) |
| PhSO₂NHNH₂ | (17.2) |
| PhNHNHPh | (26.1) |

HYDROXAMIC ACID

| | | |
|---|---|---|
| 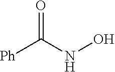 | [symbol] . [symbol] 8 (NH) | (13.7) |

AMIDINES

| | |
|---|---|
| 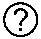 | |
| R = Me | (17.3) |
| Ph | (15.0) |

PROTONATED HETEROCYCLES

| | | | |
|---|---|---|---|
| DBU | 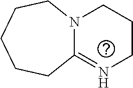 [SMILES: C1CCC2=NCCCN2CC1] | | (12) (estimate) |
| DMAP | | | |
| 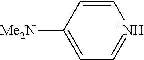 | 9.2 | 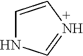 | 6.95 |
| 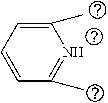 | | | |
| R = H (PPTS) | | 5.21 | (3.4) |
| t-Bu | | 4.95 | (0.90) |
| Me | | 6.75 | (4.46) |
| Cl, H | | 0.72 | |

HETEROCYCLES

| | | | |
|---|---|---|---|
| 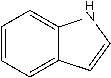 | (20.95) | | |
| 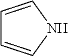 | (23.0) | 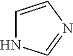 | (18.6) |
| 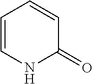 | (17.0) | 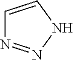 | (13.9) |

*Values <0 for H₂O and DMSO, and values >14 for water and >35 for DMSO were
extrapolated using various methods. [symbol]
indicates data missing or illegible when filed

| | | | |
|---|---|---|---|
| Substrate | pKa | H₂O | (DMSO) |

HYDROCARBONS

| | | |
|---|---|---|
| (Me)₃CH | 53 | |
| (Me)₂CH₂ | 51 | |
| CH₂═CH₂ | 50 | |
| CH₄ | 48 | (56) |
| Δ | 46 | |
| CH₂═CHCH₃ | 43 | (44) |
| PhH | 43 | |
| PhCH₃ | 41 | (43) |
| Ph₂CH₂ | 33.5 | (32.2) |
| Ph₃CH | 31.5 | (30.6) |
| HCCH | 24 | |
| PhCCH | 23 | (28.8) |
| XC₆H₄CH₃ | | |
| X = p-CN | | (30.8) |
| p-NO₂ | | (20.4) |
| p-COPh | | (26.9) |
| 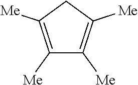 | | (26.1) |
| 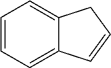 | 20 | (20.1) |
| 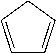 | 15 | (18.0) |
| H₂ | −36 | |

ESTERS

| | | |
|---|---|---|
| 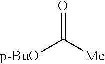 | 24.5 | (30.3) |
| 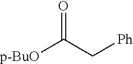 | | (23.6) |
| 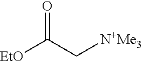 | | (20.0) |
| 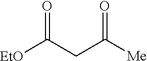 | 11 | (14.2) |
| 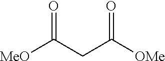 | 13 | (15.7) |
| 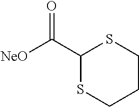 | | (20.9) |
| 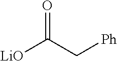 | [30.2 (THF)] | |

AMIDES

| | |
|---|---|
| 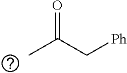 | (26.6) |
| 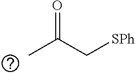 | (25.9) |
| 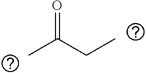 | (24.9) |
| 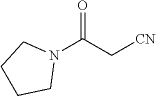 | (17.2) |
| 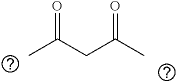 | (18.2) |
| 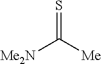 | (25.7) |

KETONES

| | | |
|---|---|---|
| 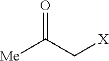 | | |
| X = H | | (26.5) |
| Ph | | (19.8) |
| SPh | | (18.7) |
| COCH₃ | 9 | (13.3) |
| SO₂Ph | | (15.1) |
| 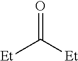 | 19-20 | (27.1) |
| 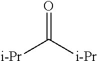 | | (28.3) |
| 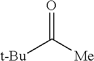 | | (27.7) |
| 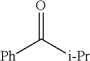 [SMILES: CC(C)C(=O)c1ccccc1] | | (26.3) |
| 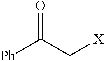 | | |
| X = H | | (24.7) |
| CH₃ | | (24.4) |
| Ph | | (17.7) |
| COCH₃ | | (12.7) |
| COPh | | (13.3) |
| CO₂Et | | (22.7) |
| CN | | (10.2) |
| F | | (21.6) |
| OMe | | (22.85) |
| OPh | | (21.1) |
| SPh | | (16.9) |
| SePh | | (18.6) |
| NPh₂ | | (20.3) |
| N⁺Me₃ | | (14.6) |
| NO₂ | | (7.7) |
| SO₂Ph | | (11.4) |
| 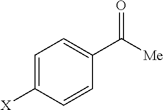 | | |
| X = H | | (24.7) |
| OMe | | (25.7) |
| NMe₂ | | (27.5) |
| Br | | (23.8) |
| CN | | (22.0) |
| 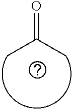 | | |
| n = 4 | | (25.1) |
| 5 | | (25.8) |
| 6 | | (26.4) |
| 7 | | (27.7) |
| 8 | | (27.4) |
| 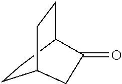 | | (28.1) |
| 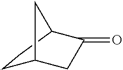 | | (29.0) |
| 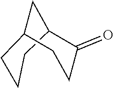 | | (25.5) |
| 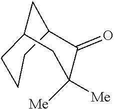 [SMILES: CC1(C)CC2CCCC(C2)C1=O] | | (32.4) |

*Values <0 for H₂O and DMSO, and values >14 for water and >35 for DMSO were
extrapolated using various methods.

| | | | |
|---|---|---|---|
| Substrate | pKa | H₂O | (DMSO) |

NITRILES

| | | |
|---|---|---|
| 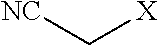 | | |
| X = H | | (31.3) |
| CH₃ | | (32.5) |
| Ph | | (21.9) |
| COPh | | (10.2) |
| CONH₂ | | (17.1) |
| CO₂Et | | (13.1) |
| CN | 11 | (11.1) |
| OPh | | (28.1) |
| N⁺Me₃ | | (20.6) |
| SPh | | (20.8) |
| SO₂Ph | | (12.0) |

HETERO-AROMATICS

| | |
|---|---|
| 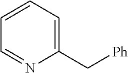 [SMILES: c1ccc(Cc2ccccn2)cc1] | (28.2) |
| 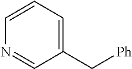 [SMILES: c1ccc(Cc2cccnc2)cc1] | (30.1) |
| 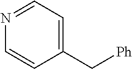 [SMILES: c1ccc(Cc2ccncc2)cc1] | (26.7) |
| 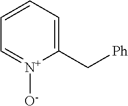 [SMILES: [O-][n+]1ccccc1Cc1ccccc1] | (25.2) |
| 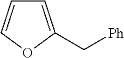 [SMILES: c1ccc(Cc2ccco2)cc1] | (30.2) |
| 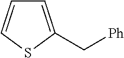 [SMILES: c1ccc(Cc2cccs2)cc1] | (30.0) |

SULFIDES

| | | |
|---|---|---|
| PhSCH₂X | | |
| X = Ph | | (30.8) |
| CN | | (20.8) |
| COCH₃ | | (18.7) |
| COPh | | (16.9) |
| NO₂ | | (11.8) |
| SPh | | (30. [symbol] ) |
| SO₂Ph | | (20.3) |
| SO₂CF₃ | | (11.0) |
| POPh₂ | | (24.9) |
| MeSCH₂SO₂Ph | | (23.4) |
| PhSCHPh₂ | | (2 [symbol] .7) |
| (PhS)₃CH | | (22.8) |
| (PrS)₃CH | | (31.3) |
| 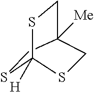 | | (30.5) |
| (PhS)₂CHPh | | (23.0) |
| 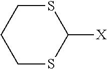 | | |
| X = Ph | | (30.7) |
| CO₂Me | | (20.8) |
| CN | | (19.1) |
| RSCH₂CN | | |
| R = Me | | (24.3) |
| Et | | (24.0) |
| i-Pr | | (23. [symbol] ) |
| t-Bu | | (22.9) |
| PhSCH═CHCH₂SPh | | (26.3) |
| BuSH | 10-11 | (17.0) |
| PhSH | −7 | (10.3) |

SULFOXIDES

| | |
|---|---|
| 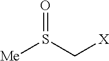 | |
| X = H | (35.1) |
| Ph | (29.0) |
| SPh | (29.0) |
| 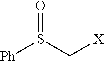 | |
| X = H | (33) |
| Ph | (27.2) |
| SOPh | (1 [symbol] .2) |
| 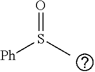 | (24.5) |

SULFONIUM

| | |
|---|---|
| 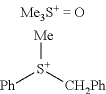 | (16.2) (16.3) |

SULFIMIDES & SULFOXIMINES

| | |
|---|---|
| 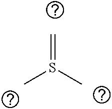 | |
| R = Me | (27.6) |
| i-Pr | (30.7) |
| 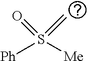 | (24.5) |
| 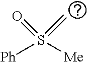 | (33) |
| 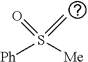 | (14.4) |
| 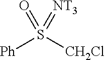 | (20.7) |

SULFONES

| | |
|---|---|
| 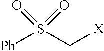 | |
| X = H | (29.0) |
| CH₃ | (31.0) |
| t-Bu | (31.2) |
| Ph | (23.4) |
| CH═CH₂ | (22.5) |
| CH═CHPh | (20.2) |
| CCH | (22.1) |
| CCPh | (17. [symbol] ) |
| COPh | (11.4) |
| COMe | (12.5) |
| OPh | (27.9) |
| N⁺Me₃ | (19.4) |
| CN | (12.0) |
| NO₂ | (7.1) |
| SMe | (23.5) |
| SPh | (20.5) |
| SO₂Ph | (12.2) |
| PPh₂ | (20.2) |
| 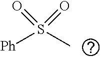 | (22.3) |
| 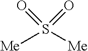 | (31.1) |
| 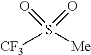 | (18.8) |
| 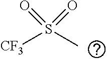 | (21. [symbol] ) |
| 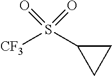 | (26.6) |
| 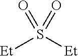 | (32.8) |
| (PhSO₂)₃CH₂Me | (14.3) |

*Values <0 for H₂O and DMSO, and values >14 for water and >35 for DMSO were
extrapolated using various methods. [symbol]
indicates data missing or illegible when filed

| | | | |
|---|---|---|---|
| Substrate | pKa | H₂O | (DMSO) |

ETHERS

| | |
|---|---|
| CH₃OPh | (49) |
| MeOCH₂SO₂Ph | (30.7) |
| PhOCH₂SO₂Ph | (27.9) |
| PhOCH₂CN | (2 [symbol] .1) |
| 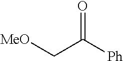 [SMILES: COCC(=O)c1ccccc1] | (21.1) |

SELENIDES

| | |
|---|---|
| 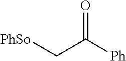 | (18.6) |
| PhSeCHPh₂ | (27.5) |
| (PhSe)₂CH₂ | (31.3) |
| PhSeCH₂Ph | (31.0) |
| PhSeCH═CHCH₂SePh | (27.2) |

AMMONIUM

| | |
|---|---|
| Me₃N⁺CH₂X | |
| X = CN | (20.6) |
| SO₂Ph | (19.4) |
| COPh | (14.6) |
| CO₂Et | (20.6) |
| CONEt₂ | (24.9) |

PHOSPHONIUM

| | |
|---|---|
| P⁺H₄ | −14 |
| MeP⁺H₃ | 2.7 |
| Et₃P⁺H | 9.1 |
| Ph₃P⁺CH₃ | (22.4) |
| Ph₃P⁺i-Pr | (21.2) |
| Ph₃P⁺CH₂COPh | (6.2) |
| Ph₃P⁺CH₂CN | (7.0) |

PHOSPHONATES &
PHOSPHINE OXIDES

| | |
|---|---|
| 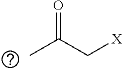 | |
| X = Ph | (27.6) |
| CN | (16.4) |
| CO₂Et | (18.6) |
| Cl | (26.2) |
| SiMe₃ | (2 [symbol] . [symbol] ) |
| 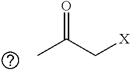 | |
| X = SPh | (24.9) |
| CN | (16.9) |

PHOSPHINES

| | |
|---|---|
| Ph₂PCH₂PPh₂ | (29.9) |
| Ph₂PCH₂SO₂Ph | (20.3) |

NITRO

| | | |
|---|---|---|
| RNO₂ | | |
| R = CH₃ | −10 | (17.2) |
| CH₂Me | | (16.7) |
| CHMe₂ | | (16.9) |
| CH₂Ph | | (12.2) |
| CH₂Bn | | (16.2) |
| CH₂SPh | | (11. [symbol] ) |
| CH₂SO₂Ph | | (7.1) |
| CH₂COPh | | (7.7) |
| 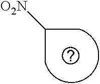 | | |
| n = 3 | | (26.9) |
| 4 | | (17.8) |
| 5 | | (16.0) |
| 6 | | (17.9) |
| 7 | | (15.8) |

IMINES

| | |
|---|---|
| 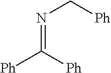 | (24.3) |

Oxime ethers are- 10 pka units less
acidic than their ketone counterparts
Str [symbol] wieser, JOC 1991, [symbol] , 1989
*Values <0 for H₂O and DMSO, and values >14 for water and >35 for DMSO were
extrapolated using various methods.
REFERENCES
DMSO:
JACS 97, 7007 (1975)
JACS 97, 7160 (1975)
JACS 97, 442 (1975)
JACS 105, 6188 (19+Z,899 3)
JOC 41, 1863 (1976)
JOC 41, 1885 (1976)
JOC 41, 2766 (1976)
JOC 41, 2508 (1976)
JOC 42, 1817 (1977)
JOC 42, 321 (1977)
JOC 42, 326 (1977)
JOC 43, 3113 (197+Z,899 )
JOC 43, 3096 (1978)
JOC 43, 1764 (197+Z,899 )
JOC 45, 3325 (1980)
JOC 45, 3305 (1980)
JOC 45, 3884 (1980)
JOC 46, 4327 (1981)
JOC 46, 632 (1981)
JOC 47, 3224 (1982)
JOC 47, 2504 (1982)
Acc. Chem. Res. 21, 456 (1988)
Unpublished results of F. Bordwell
Water:
Advanced Org. Chem., 3rd Ed
J. March (1985)
Unpublished results of W. P. Jenc+z,899
THF:
JACS 110, 5705 (1988) [symbol]
indicates data missing or illegible when filed

By a “soft nucleophile” is meant a nucleophile with a large, polarizable orbital with low charge density that can overlap with the orbital of the nucleus of the other molecule, to form the covalent bond. A “soft nucleophile,” as used herein, is a nucleophile that can undergo reversible addition to a ketone or oxocarbenium ion. Accordingly, a suitable soft nucleophile should be sufficiently nucleophilic to undergo addition to a ketone or oxocarbenium ion and also be a sufficiently good leaving group to make the addition reversible. In some embodiments, a soft nucleophile, as used herein, may have a pKa/pKaH between about 0 and about 20, or any value in between, such as 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5 or 20. By “about” is meant a variance (plus or minus) from a value or range of 5% or less, for example, 0.5%, 1%, 1.5%, 2.0%, 2.5%, 3.0%, 3.5%, 4.0%, 4.5%, 5.0%, etc. It is to be understood that the pKas of functional groups such as alkynes or carbonyl compounds can be modified by the addition of a Lewis acid; examples of such functional groups include but are not limited to: water, alcohols, hydrogen sulfide, thiols, fluoride, hypochlorite, cyanide, azide, thiocyanide, bisulfite, phosphates, nitrites, ammonia, amines, boric acid, boronic acids, borinic acids, hydrogen peroxide, carboxylic acids, oximes, hydroxamic acids, hydroperoxides, sulfinic acids, imides, sulfonamides, guanidines, hydrazides, hydrazine, hydrazines, amidines, imidazoles, 1,2,3-triazoles, pyridones, indene, cyclopentadiene, 1,3-dicarbonyls, malononitrile, 3-oxonitriles, sulfoniums, phosphoniums, stabilised phosphonates and nitroalkanes. It is also to be understood that suitable soft nucleophiles may have pKas outside the range 0 and 20 but can undergo reversible addition to a ketone or oxocarbenium ion. Soft nucleophiles include, without limitation, MeCN, N₃—, CN—, MeNO₂ etc. By contrast, a “hard” nucleophile is a highly reactive atom or molecule, such as a Grignard reagent or alkyl lithium, and is specifically excluded. As used herein, “Nu” refers to a soft nucleophile that may attack the halohydrin ketone compound prior to cyclization, or may be attached to the oxonium ion resulting from cyclization of the halohydrin ketone compound.

By “halohydrin ketone compound” is meant a compound containing a ketone group and functional group in which a halogen and a hydroxyl are bonded to adjacent groups. Accordingly, in some embodiments, the halohydrin ketone compound may have the following general structure, where X may be a halogen, NB may be optionally substituted aryl, arylalkyl, heteroaryl, or heteroarylalkyl and R₁ and R₂ may each independently be H, alkyl, ester, or protecting group:

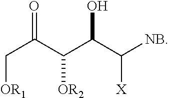

In some embodiments, R₁ and R₂ may each independently be OH, —OC(CH₃)₂O—, —(CH₂)₃—, —CH₂SCH₂—, or —CH₂OCH₂—.

In some embodiments, the halohydrin ketone compound may have the following general structure, where X may be a halogen, NB may be optionally substituted aryl, arylalkyl, heteroaryl, or heteroarylalkyl, Y may be CH₂, O, S, NR, where R may be alkyl, aryl, acyl, or carboxylate (for example, tert-butyloxycarbonyl, benzyloxycarbonyl, or propargyl), and Z may be a protecting group for an alcohol, including without limitation, acetonide, silyl protecting group, alkyl protecting group or aryl protecting group (including cyclic or acyclic), for use as an intermediate in the synthesis of a nucleoside or analogue thereof:

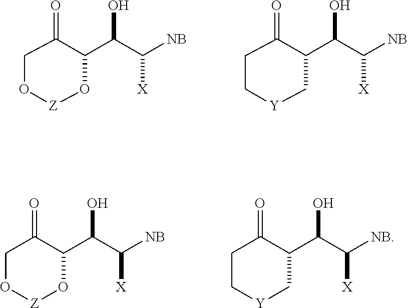

In some embodiments, the halohydrin ketone compound may have the following general structure, where NB may be optionally substituted aryl, arylalkyl, heteroaryl, or heteroarylalkyl and X may be a halogen:

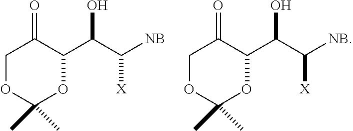

In some embodiments, the halohydrin ketone compound may have the following general structure, where NB is optionally substituted aryl, arylalkyl, heteroaryl, or heteroarylalkyl, X is a halogen, and Y may be CH₂, O, S, NR, where R may be alkyl, aryl, acyl, or carboxylate (for example, R may be tert-butyloxycarbonyl, benzyloxycarbonyl, or propargyl):

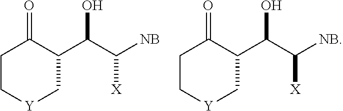

By “nucleoside” is meant a glycosylamine having a nitrogenous base or “nucleobase” or “NB” and a sugar ring (e.g., ribose or deoxyribose), in which the anomeric carbon is linked through a glycosidic bond to the N9 of a purine (e.g., adenine or guanine) or the N1 of a pyrimidine (e.g., cytosine, thymine, or uracil). Nucleosides include both L- and D-nucleoside isomers. Examples of nucleosides include cytidine, uridine, adenosine, guanosine, thymidine and inosine.

Nucleoside analogues (NAs) are compounds that are structurally similar to naturally occurring nucleosides. NAs may include, without limitation, compounds with modifications at positions C1′, C2′, C3′, C4′ and/or C5′ of the sugar ring, based on conventional numbering as exemplified:

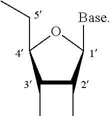

In some embodiments, NAs may exist as a free triol or may be phosphorylated at C3′ and/or C5′. In some embodiments, NAs may include, without limitation, compounds with a saturated or unsaturated carbocyclic ring. In some embodiments, NAs may include nitrogen in the sugar ring, for example as a replacement for the naturally occurring oxygen, and/or may include N—R groups, where R may be without limitation alkyl, allyl, alkynyl or benzyl. In some embodiments, the nucleoside analogues disclosed herein may be modified to function as a phosphoramidate or phosphonamidate compound, e.g., a “ProTide,” which includes a 5′-nucleoside monophosphate in which the two hydroxyl groups are masked with an amino acid ester and an aryloxy component which can be enzymatically metabolized to deliver free 5′-monophosphate, which is further transformed to the active 5′-triphosphate form of the nucleoside analogue, once delivered to a cell. In some embodiments, NAs that include sulphur in the sugar ring, for example as a replacement for the naturally occurring oxygen, are specifically excluded.

The “NB” or nucleobase or base of NAs may be an aryl, arylalkyl, heteroaryl, or heteroarylalkyl attached from the C1 position to a carbon or nitrogen atom. NBs may also be modified, for example, may be any one or more of 5,6-dihydrouracil, 5-methylcytosine, 5-hydroxymethylcytosine, 5,5,5-trifluoromethylthymine, 5-fluorouracil, 2-thiouracil, 4-methylbenzimidazole, hypoxanthine, 7-deazaguanine, 7-deazaadenine, indole, imidazole, triazole, pyrrole, pyrazole, etc. It is to be understood that enantiomers of aldol products (halohydrins) can be produced using D-proline catalysis and may be used to prepare enantiomeric NAs.

“Alkyl” refers to a straight or branched hydrocarbon chain group consisting solely of carbon and hydrogen atoms, generally containing no unsaturation and including, for example, from one to ten carbon atoms, or any value in between, such as 1, 2, 3, 4, 5, 6, 7, 8, 9 or 10 carbon atoms, and which is attached to the rest of the molecule by a single bond.

In some embodiments, alkyl may refer to a straight or branched hydrocarbon chain group consisting solely of carbon and hydrogen atoms, generally containing no unsaturation and including from one to six carbon atoms, or any value in between, such as 1, 2, 3, 4, 5, or 6 carbon atoms, and which is attached to the rest of the molecule by a single bond. Unless stated otherwise specifically in the specification, the alkyl group may be optionally substituted by one or more substituents as described herein. Unless stated otherwise specifically herein, it is understood that the substitution can occur on any carbon of the alkyl group.

By “aryl” is meant a monocyclic or bicyclic aromatic ring containing only carbon atoms, including for example, 5-14 members, such as 5, 6, 7, 8, 9, 10, 11, 12, 13, or 14 members. Examples of aryl groups include phenyl, biphenyl, naphthyl, indanyl, indenyl, tetrahydronaphthyl, 2,3-dihydrobenzofuranyl, dihydrobenzopyranyl, 1,4-benzodioxanyl, and the like. Unless stated otherwise specifically herein, the term “aryl” is meant to include aryl groups optionally substituted by one or more substituents as described herein.

“Heteroaryl” refers to a single or fused aromatic ring group containing one or more heteroatoms in the ring, for example N, O, S, including for example, 5-14 members, such as 5, 6, 7, 8, 9, 10, 11, 12, 13, or 14 members. Examples of heteroaryl groups include furan, thiophene, pyrrole, oxazole, thiazole, imidazole, pyrazole, isoxazole, isothiazole, 1,2,3-oxadiazole, triazole (e.g., 1,2,3-triazole or 1,2,4-triazole), 1,3,4-thiadiazole, tetrazole, pyrazole, pyridine, pyridazine, pyrimidine, 2,6-dichloropyrimidine pyrazine, 1,3,5-triazine, imidazole, benzimidazole, benzoxazole, benzothiazole, indolizine, indole, isoindole, benzofuran, benzothiophene, 1 H-indazole, purine, 4H-quinolizine, quinoline, isoquinoline, cinnoline, phthalazine, quinazoline, quinoxaline, 1,8-naphthyridine, pteridine, uracil, thymine, deazadenine, phthalimide, adenine, and the like. Unless stated otherwise specifically herein, the term “heteroaryl” is meant to include heteroaryl groups optionally substituted by one or more substituents as described herein.

By “arylalkyl” is meant a group of the formula —RₐR_{b} where Rₐ is a C₁₋₁₀ alkyl group as described herein and R_{b} is one or more aryl moieties as described herein. The arylalkyl group(s) may be optionally substituted as described herein. Examples of arylalkyl groups include without limitation benzyl, arylalkyl groups include without limitation benzyl, phenethyl, phenylpropyl, (4-methylphenyl)methyl, (4-methylphenyl)ethyl, (2-methylphenyl)methyl, (2,4,6-trimethylphenyl), (4-fluorophenyl)methyl, (4-trifluoromethylphenyl)methyl, etc.

“Heteroarylalkyl” refers to a group of the formula —RₐR_{c} where Rₐ is a C₁₋₁₀ alkyl group as described herein and Rₑ is one or more heteroaryl moieties as described herein. The heteroarylalkyl group(s) may be optionally substituted as described herein. Examples of heteroarylalkyl groups include without limitation, furanylmethyl, thiphenylmethyl, pyridylmethyl, imidazolylmethyl, uridinylmethyl, etc.

“Acyl” refers to a group of the formula —C(O)Rₐ, where Rₐ is a C₁₋₁₀ alkyl or a C₁₋₆ alkyl group as described herein. The alkyl group may be optionally substituted as described herein.

By “ester” is meant a carbon atom double bonded to a first oxygen atom and single bonded to a second oxygen atom (A-COOA′), where A can be any carbon linked group.

Suitable ester groups include without limitation, methyl, ethyl, propyl, iso-propyl, butyl, iso-butyl, sec-butyl, pentyl, etc.

By “cyclic” is meant a group in which one or more series of atoms are connected to form a ring group. The atoms in a cyclic group may be carbon, oxygen, nitrogen, silicon or sulfur. The ring may vary in size and include for example, 3 or 8 atoms, or any value in between and inclusive, such as 3, 4, 5, 6, 7, or 8. Suitable cyclic groups include without limitation, 1,3-dioxolane, 1,3-dioxanone, oxazoline, tetrahydrofuran, etc.

Halogens include bromine, chlorine, fluorine, iodine, etc. and are represented by “X” in the chemical structures disclosed herein. In some embodiments, a halogen may include chlorine or fluorine. In some embodiments, the halogen may be fluorine. In some embodiments, the halogen may be chlorine. Accordingly, “halo” refers to bromo, chloro, fluoro, iodo, etc. A halide is a halogen atom bearing a negative charge.

A “protecting group” as used herein, means a reversibly formed derivative of an existing functional group in a molecule that is temporarily attached to decrease reactivity such that the protected functional group does not react under synthetic conditions to which the molecule is subjected in one or more subsequent steps. Protecting groups are as known in the art and described herein. It is to be understood that a person skilled in the art will readily be able to determine a suitable protecting group for a particular synthesis.

In some embodiments, a “suitable alcohol protecting group” or an “alcohol protecting group” includes, without limitation, acetonide, silyl protecting group, alkyl protecting group or aryl protecting group (including cyclic or acyclic), such as a silyl ether for example, tert-butyl(dimethyl)sily/(TBS), triisopropylsilyl (TIPS), trimethylsilyl (TMS), tert-butyldiphenylsilyl (TBDPS), triethylsilyl (TES), 2-(trimethylsilyl)ethoxymethyl ether (SEM), etc.; acetate (Ac), pivalate (Piv) or other ester or carbonate protecting group etc.; benzyl, allyl, methoxymethyl or p-methoxybenzyl (PMB) or other ether protecting group, etc.; tetrahydropyranyl (THP) ether or other related protecting groups, etc.

By “covalent bond to Nu” is meant the conjugation of an “R” group as described herein, e.g., R, R₁, R₂, R₃, or R₄, to a soft nucleophile as described herein. Examples of such conjugations include, without limitation, attack of the C3′ or C5′ hydroxyl into the soft nucleophile to form a covalent bond, such as attack on a nitrilium group to form an oxazoline.

“Optional” or “optionally” means that the subsequently described event of circumstances may or may not occur, and that the description includes instances where the event or circumstance occurs one or more times and instances in which it does not. For example, “optionally substituted alkyl” means that the alkyl group may or may not be substituted and that the description includes both substituted alkyl groups and alkyl groups having no substitution, and that the alkyl groups may be substituted one or more times. Examples of optionally substituted alkyl groups include, without limitation, methyl, ethyl, propyl, butyl, pentyl, hexyl, isopropyl, isobutyl, sec-butyl, tert-butyl, etc. Examples of suitable optional substituents include, without limitation, H, F, Cl, CH₃, OH, OCH₃, CF₃, CHF₂, CH₂F, CN, halo, and C₁₋₁₀ alkoxy. Similarly, “optionally substituted aryl- or heteroaryl-” mean aryl-or heteroaryl-groups that may or may not be substituted and that the description includes both substituted aryl- or heteroaryl-groups and aryl- or heteroaryl-groups having no substitution, and that the aryl- or heteroaryl-groups may be substituted one or more times.

Examples of suitable optional substituents include, without limitation, H, F, Cl, CH₃, OH, OCH₃, CF₃, CHF₂, CH₂F, CN, halo, and C₁₋₁₀ alkoxy.

In some embodiments, the methods disclosed herein provide rapid access to intermediates in the synthesis of nucleosides or analogues thereof in good enantioselectivity and/or yield, for example, greater than about 10 g to about 400 g, or any value in between, for example 10 g, 15 g, 20 g, 25 g, 50 g, 75 g, 100 g, 125 g, 150 g, 200 g, 250 g, 300 g, 350 g, or 400 g.

Accordingly, the methods disclosed herein may be used in the process scale production of C4′-modified NAs.

In some embodiments, the present disclosure includes a method for the synthesis of a C4′ substituted nucleoside analogue for example in accordance with Scheme 1:

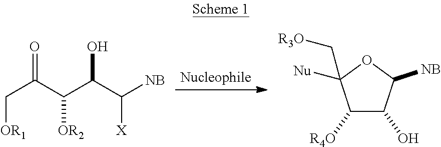

where X may be a halogen; NB may be optionally substituted aryl, arylalkyl, heteroaryl, or heteroarylalkyl; R₁ and R₂ may each independently be H, alkyl, ester, or protecting group, or may or together form a cyclic group; Nucleophile and Nu may be a soft nucleophile as described herein; R₃ may be H, alkyl, ester, protecting group, or a covalent bond to Nu; and R₄ may be H, alkyl, ester, protecting group, or a covalent bond to Nu.

In some embodiments, the present disclosure includes a method for the synthesis of a C4′ substituted nucleoside analogue for example in accordance with Scheme 2:

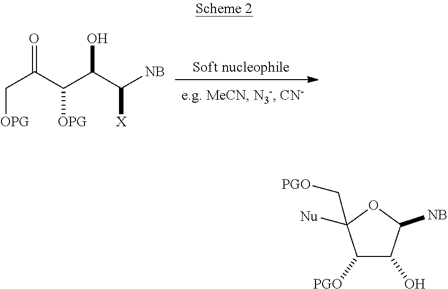

where X may be a halogen; NB may be optionally substituted aryl, arylalkyl, heteroaryl, or heteroarylalkyl; Nu may be a soft nucleophile as described herein; PG may be a protecting group that may form a covalent bond to Nu in the product. In some embodiments, the protecting group may be acetonide (CH3)2C, siloxane —Si(CH3)2, O—Si(CH3)2—, benzyl, p-methoxybenzyl, TBS, or other silyl protecting groups.

In some embodiments, the methods disclosed herein provides direct access to an array of C4′-modified NAs including, without limitation, locked nucleic acids (LNAs).

In some embodiments, a C4′ substituted nucleoside analogue that includes a nitrile moiety can be reduced, resulting in an aldehyde product.

In some embodiments, a C4′ substituted nucleoside analogue in accordance with the present disclosure includes:

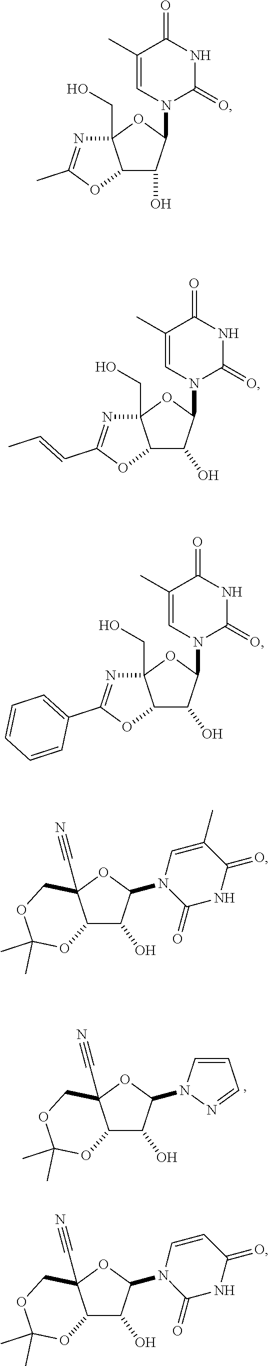

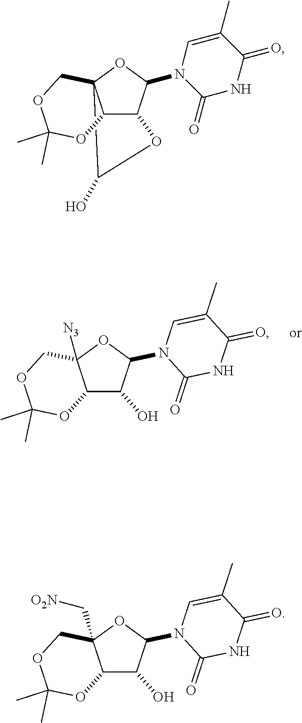

In some embodiments, the methods disclosed herein may be useful in drug design.

In some embodiments, the nucleoside analogues disclosed herein may be used as small molecule therapeutics or as monomers in oligonucleotide therapeutics.

In some embodiments, the methods disclosed herein may be useful in the preparation of diversity libraries. For example, larger collections of C4′-modified NAs (e.g., focused screening libraries) can be generated using the methods described herein.

As used herein the singular forms “a”, “and”, and “the” include plural referents unless the context clearly dictates otherwise. For example, “a compound” refers to one or more of such compounds. Throughout this application, it is contemplated that the term “compound” or “compounds” refers to the compounds discussed herein and includes precursors and derivatives of the compounds. The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention, unless specifically indicated otherwise. Any formulas, structures or names of compounds described in this specification that do not specify a particular stereochemistry are meant to encompass any and all existing isomers as described above and mixtures thereof in any proportion.

When stereochemistry is specified, the invention is meant to encompass that particular isomer in pure form or as part of a mixture with other isomers in any proportion. Single enantiomers, i.e., optically active forms, can be obtained by asymmetric synthesis or by resolution of the racemates. Resolution of the racemates can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. These procedures can be repeated, if desired, to increase the enantiomeric purity of a compound. When the compounds described herein contain olefmic double bonds or other centers of geometric asymmetry, and unless otherwise specified, it is intended that the compounds include the cis, trans, Z- and E-configurations. Likewise, all tautomeric forms are also intended to be included.

The starting materials can be obtained from commercial sources, prepared from commercially available organic compounds, prepared using known synthetic methods.

The present invention will be further illustrated in the following examples.

### Materials and Methods

Nuclear magnetic resonance (NMR) spectra were recorded using deuterochloroform (CDCl₃), deuteromethanol (CD₃OD), deuteroacetone ((CD₃)₂CO), deuteroacetonitrile (CD₃CN) or deuterodimethyl sulfoxide (DMSO-d₆) as the solvent. Signal positions (6) are given in parts per million from tetramethylsilane (δ 0) and were measured relative to the signal of the solvent (¹H NMR: CDCl₃: δ 7.26; CD₃OD: δ 3.31; (CD₃)₂CO: δ 2.05; CD₃CN: δ 1.96; DMSO-d₆: δ 2.50; ¹³C NMR: CDCl₃: δ 77.16; CD₃OD: δ 49.00; (CD₃)₂CO: δ 29.84; CD₃CN: δ 1.32; DMSO-d₆: 39.5). Coupling constants (J values) are given in Hertz (Hz) and are reported to the nearest 0.1 Hz. ¹H NMR spectral data are tabulated in the order: multiplicity (s, singlet; d, doublet; t, triplet; q, quartet; sept, septet; m, multiplet; br broad), coupling constants, number of protons. NMR spectra were recorded on a Bruker Avance 600 equipped with a QNP or TCl cryoprobe (600 MHz), Bruker 400 (400 MHz) or Bruker 500 (500 MHz). Diastereomeric ratios (dr) are based on analysis of crude ¹H NMR. Assignments of ¹H are based on analysis of ¹H-¹H-COSY and nOe spectra. Assignments of ¹³C are based on analysis of HSQC spectra.

### Examples

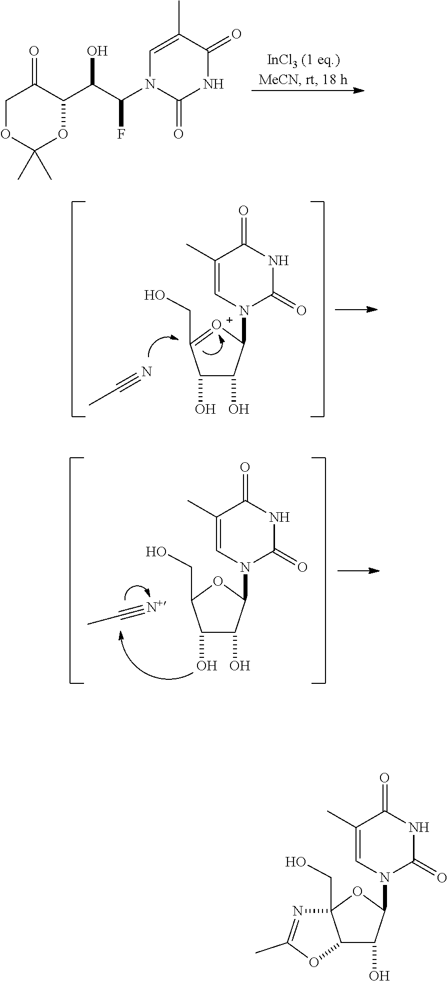

To a stirred solution of starting material ketone (50.0 mg, 158 μmol, 1 eq.) in MeCN (1.58 mL, 0.1 molar reaction concentration) was added InCl₃ (35.0 mg, 158 μmol, 1 eq.). The reaction was stirred at room temperature for 18 h, then quenched by addition of saturated aqueous sodium bicarbonate solution (2 mL). The mixture was extracted with ethyl acetate (10×5 mL) and the combined organic extracts dried (MgSO₄) and concentrated in vacuo.

The crude product was purified by silica chromatography (ethyl acetate/hexanes) to afford pure product (25.0 mg, 84.1 μmol, 53%).

¹H NMR (500 MHz, MeCN-d₆) δ 9.13 (1H, br s), 7.35 (1H, q, J=1.0 Hz), 5.65 (1H, d, J=8.2 Hz), 4.71 (1H, d, J=5.9 Hz), 4.31 (1H, m), 3.80 (1H, dd, J=11.8, 5.2 Hz), 3.71 (1H, d, J=7.8 Hz), 3.60 (1H, dd, J=11.8, 6.3 Hz), 3.36 (1H, app t, J=6.1 Hz), 2.01 (3H, s), 1.87 (3H, d, J=1.0 Hz).

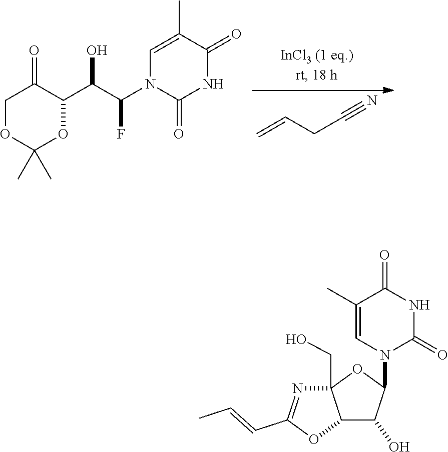

To a stirred solution of starting material ketone (5.00 mg, 15.8 μmol, 1 eq.) in allylnitrile (0.32 mL, 0.05 molar reaction concentration) was added InCl₃ (3.5 mg, 15.8 μmol, 1 eq.). The reaction was stirred at room temperature for 18 h, then quenched by addition of saturated aqueous sodium bicarbonate solution (1 mL). The mixture was extracted with ethyl acetate (5×2 mL) and the combined organic extracts dried (MgSO₄) and concentrated in vacuo. The crude product was purified by silica chromatography (ethyl acetate/hexanes) to afford pure product.

¹H NMR (500 MHz, MeCN-d₆) δ 9.05 (1H, br s), 7.35 (1H, s), 6.89 (1H, dq, J=15.8, 6.9 Hz), 6.08 (1 H, dq, J=15.8, 1.6 Hz), 5.57 (1H, d, J=8.4 Hz), 4.75 (1 H, d, J=5.8), 4,32 (1H, m), 3.83 (1H, dd, J=11.9, 3.0 Hz), 3.75 (1H, br d, J=6.3 Hz), 3.63 (1H, m), 3.36 (1 H, br t, J=5.1 Hz), 1.94 (3H, dd, J=6.9, 1.6 Hz), 1.88 (3H, d, J=0.9 Hz).

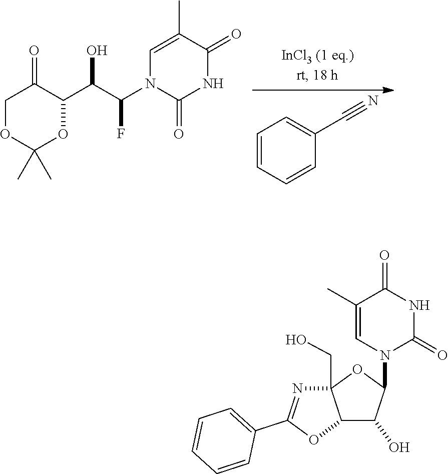

To a stirred solution of starting material ketone (5.00 mg, 15.8 μmol, 1 eq.) in benzonitrile (0.32 mL, 0.05 molar reaction concentration) was added InCl₃ (3.5 mg, 15.8 μmol, 1 eq.). The reaction was stirred at room temperature for 18 h, then quenched by addition of saturated aqueous sodium bicarbonate solution (1 mL). The mixture was extracted with ethyl acetate (5×2 mL) and the combined organic extracts dried (MgSO₄) and concentrated in vacuo. The crude product was purified by silica chromatography (ethyl acetate/hexanes) to afford pure product.

¹H NMR (500 MHz, MeCN-d₃) δ 8.06 (2H, d, J=7.6 Hz), 7.65 (1H, t, J=7.6 Hz), 7.55 (2H, t, J=7.6 Hz), 7.38 (1H, s), 5.66 (1H, d, J=8.4 Hz), 4.96 (1H, d, J=5.7 Hz), 4.29 (1H, m), 3.93 (1H, m), 3.73 (1H, br d, J=11.7 Hz), 1.87 (3H, s).

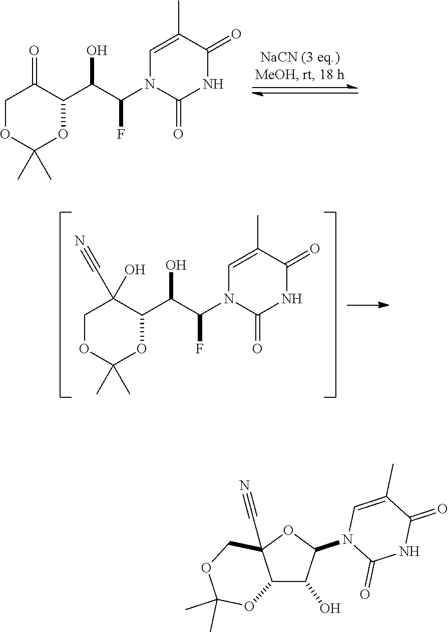

To a stirred solution of starting material ketone (100.0 mg, 316 μmol, 1 eq.) in MeOH (6.3 mL, 0.05 molar reaction concentration) was added NaCN (46.49 mg, 949 μmol, 3 eq.). The reaction was stirred at room temperature for 18 h then was concentrated in vacuo. The crude product was purified by silica chromatography (ethyl acetate/hexanes) to afford pure product (70.0 mg, 0.22 mmol, 68%).

¹H NMR (500 MHz, MeOH-d₄) δ 7.32 (1H, s), 5.90 (1H, br s), 4.61 (1H, br d, J=5.7 Hz), 4.38 (1H, d, J=5.7 Hz), 4.27 (2H, abq, J=10.1 Hz), 1.90 (3H, s), 1.64 (3H, s), 1.58 (3H, s).

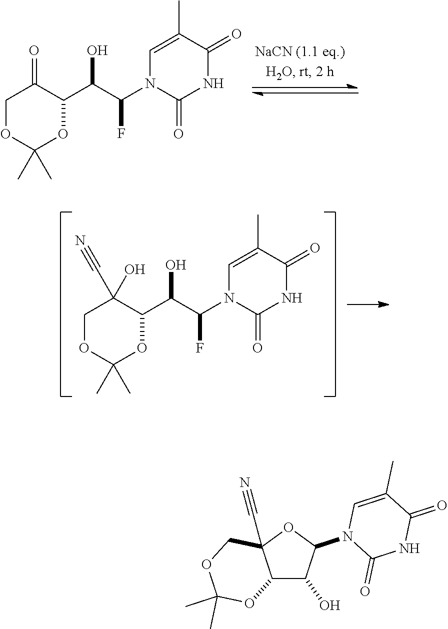

To a stirred solution of starting material ketone (1.00 g mg, 3.16 mmol, 1 eq.) in water (63.2 mL, 0.05 molar reaction concentration) was added NaCN (170 mg, 3.48 mmol, 1.1 eq.). The reaction was stirred at room temperature for 2 hours, then was quenched by addition of ferrous sulfate (483 mg, 1.74 mmol, 0.55 eq.). The mixture was then filtered, and the filtrate extracted with ethyl acetate (10×50 mL). The combined organic extracts were dried (magnesium sulfate) and concentrated in vacuo. The crude product was purified by silica chromatography (ethyl acetate/hexanes) to afford pure product (492 mg, 1.52 mmol, 48%).

¹H NMR (500 MHz, MeOH-d₄) δ 7.32 (1H, s), 5.90 (1H, br s), 4.61 (1H, br d, J=5.7 Hz), 4.38 (1H, d, J=5.7 Hz), 4.27 (2H, abq, J=10.1 Hz), 1.90 (3H, s), 1.64 (3H, s), 1.58 (3H, s).

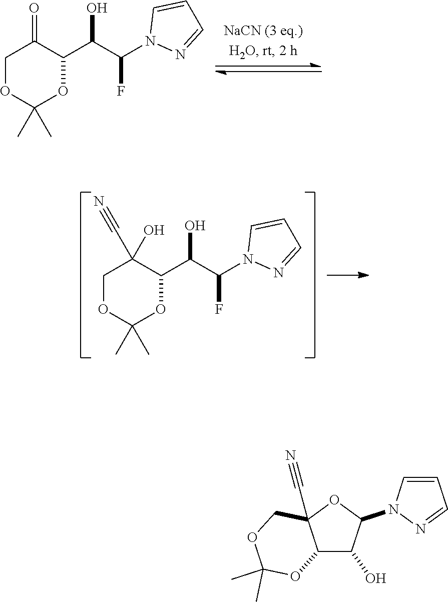

To a stirred solution of starting material ketone (5.0 mg, 19 μmol, 1 eq.) in water (0.19 mL, 0.1 molar reaction concentration) was added NaCN (2.8 mg, 58 μmol, 3 eq.). The reaction was stirred at room temperature for 2 hours, then was concentrated in vacuo. The crude product was purified by silica chromatography (ethyl acetate/hexanes) to afford pure product (4.6 mg, 17 μmol, 89%).

¹H NMR (500 MHz, MeCN-d₃) δ 7.75 (1H, d, J=2.4 Hz), 7.60 (1H, d, J=1.6 Hz), 7.32 (1H, dd, J=2.4, 1.6 Hz), 6.02 (1 H, br s), 4.70 (1H, dd, J=4.9, 3.6 Hz), 4.44 (1H, d, J=4.9 Hz), 4.28 (1H, d, J=10.2 Hz), 4.06 (1H, d, J=3.6 Hz), 4.03 (1H, d, J=1.02 Hz), 1.57 (3H, s), 1.54 (3H, s).

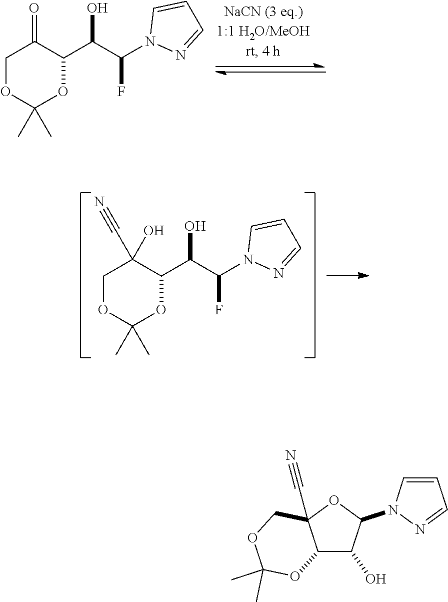

To a stirred solution of starting material ketone (5.0 mg, 19 μmol, 1 eq.) in 1:1 water/MeOH (0.19 mL, 0.1 molar reaction concentration) was added NaCN (2.8 mg, 58 μmol, 3 eq.). The reaction was stirred at room temperature for 4 hours, then was concentrated in vacuo. The crude product was purified by silica chromatography (ethyl acetate/hexanes) to afford pure product (4.6 mg, 17 μmol, 89%).

¹H NMR (500 MHz, MeCN-d₃) δ 7.75 (1H, d, J=2.4 Hz), 7.60 (1H, d, J=1.6 Hz), 7.32 (1H, dd, J=2.4, 1.6 Hz), 6.02 (1 H, br s), 4.70 (1H, dd, J=4.9, 3.6 Hz), 4.44 (1H, d, J=4.9 Hz), 4.28 (1H, d, J=10.2 Hz), 4.06 (1H, d, J=3.6 Hz), 4.03 (1H, d, J=1.02 Hz), 1.57 (3H, s), 1.54 (3H, s).

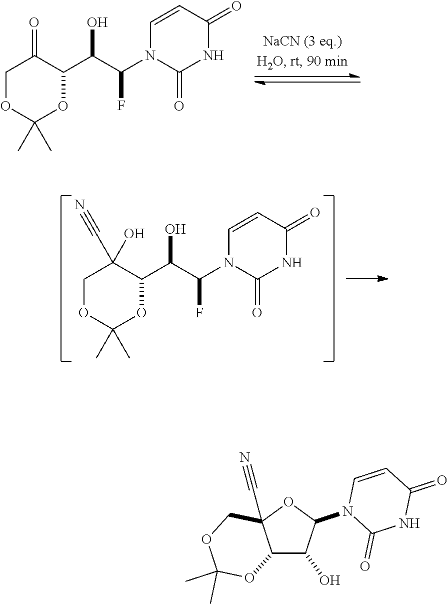

To a stirred solution of starting material ketone (5.0 mg, 17 μmol, 1 eq.) in water (0.17 mL, 0.1 molar reaction concentration) was added NaCN (2.4 mg, 50 μmol, 3 eq.). The reaction was stirred at room temperature for 2 hours, then was concentrated in vacuo. The crude product was purified by silica chromatography (ethyl acetate/hexanes) to afford pure product (4.5 mg, 15 μmol, 88%).

¹H NMR (500 MHz, MeCN-d₃) δ 9.10 (1 H, br s), 7.36 (1H, d, J=8.2 Hz), 5.83 (1 H, br s), 5.66 (1H, d, J=8.2 Hz), 4.54 (1H, br d, J=5.5 Hz), 4.31 (1H, d, J=10.2 Hz), 4.12 (1H, d, J=10.2 Hz), 4.06 (1H, d, J=5.5 Hz), 4.00 (1 H, br s) 1.55 (3H, s), 1.53 (3H, s).

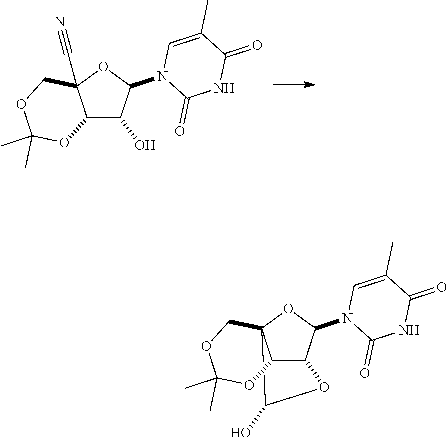

To a stirred solution of starting material nitrile (120 mg, 0.371 mmol) in dichloromethane (3.71 mL) at −78° C. was added diisobutyl aluminium hydride (1.63 mL, 0.91 molar in dichloromethane, 1.48 mmol, 4 eq.) dropwise. The reaction was stirred at −78° C. for 1 h, then quenched by addition of saturated aqueous sodium potassium tartrate (3 mL). The mixture was stirred at room temperature for 1 h, then extracted with ethyl acetate (10 mL×3). The crude material was purified by silica chromatography, eluting with hexanes/(1% isopropyl alcohol in ethyl acetate) (1:1->1:2) to afford the product as a colourless oil (12.1 mg, 3.71 mmol, 10%), along with recovered starting material (108 mg, 0.334 mmol, 90%).

¹H NMR (600 MHz, D₃CCN): δ 9.11 (s, 1H, NH), 7.38 (d, J=1.4 Hz, 1H), 5.74 (s, 1H), 5.61 (s, 1H), 4.48 (s, 1H), 4.42 (d, J=11.0 Hz, 1H), 4.01 (d, J=11.0 Hz, 1H), 3.93 (s, 1H), 1.88 (d, J=1.3 Hz, 3H), 1.51 (s, 3H), 1.43 (s, 3H); ¹³C NMR (126 MHz, D₃CCN): S 164.6, 151.0, 135.2, 111.0, 102.0, 99.5, 88.9, 80.0, 78.2, 74.5, 60.2, 29.1, 19.6, 12.5; HRMS (ESI⁺) calcd for [C₁₄H₁₈N₂O₇+H]⁺ 327.1187 m/z found: 327.1187 m/z (±0.8 ppm).

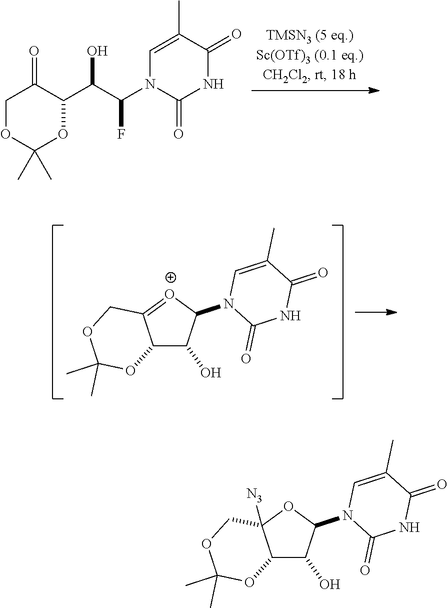

To a stirred solution of starting material ketone (5.0 mg, 16 μmol, 1 eq.) in CH₂Cl₂ (0.16 mL, 0.1 molar reaction concentration) was added TMS azide (9.1 mg, 79 μmol, 5 eq.). The reaction was stirred at room temperature for 18 h then was concentrated in vacuo. The crude product was purified by silica chromatography (ethyl acetate/hexanes) to afford pure product (3.75 mg, 12 μmol, 75%).

¹H NMR (500 MHz, MeCN-d₃) δ 7.35 (1H, q, J=1.0 Hz), 6.22 (1H, d, J=8.0 Hz), 4.67 (1H, dd, J=8.0, 4.1 Hz), 4.14 (1H, d, J=4.1 Hz), 4.11 (1H, d, J=12.8 Hz), 4.01 (1H, d, J=12.8 Hz), 1.91 (3H, d, J=1.0 Hz), 1.50 (3H, s), 1.47 (3H, s).

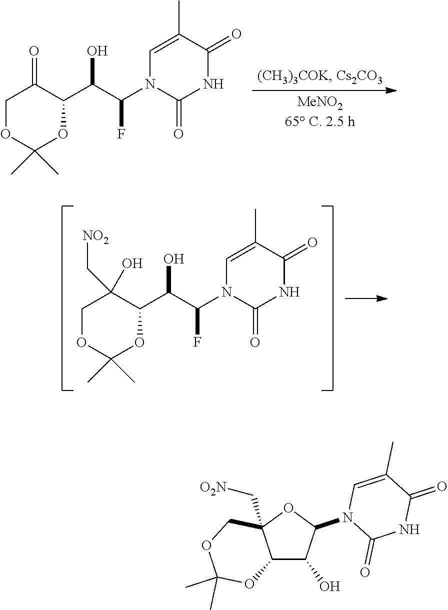

Under an argon atmosphere, potassium tert-butoxide (1.06 g, 9.48 mmol, 3 eq.) and cesium carbonate (2.06 g, 6.32 mmol, 2 eq.) was added to anhydrous nitromethane (32 mL) and heated to 65° C. for 10 minutes. After this time, a suspension formed, and the ketone (1.00 g, 3.16 mmol, 1 eq.) was added in one portion to the reaction mixture and stirred for a further 2.5 h at 65° C. Upon cooling, the reaction was quenched with sat. NH₄Cl, diluted with ethyl acetate and washed with water and brine. The organic layer was dried over NaSO₄, filtered and the solvent was removed under reduced pressure. Flash chromatography of the residue (ethyl acetate/hexanes) gave the pure product (0.621 g, 1.74 mmol, 55%).

¹H NMR (CD₃CN, 500 MHz) δ 7.19 (1H, s), 5.92 (1H, d, J=1.5 Hz), 5.31 (1H, dd, J=12.4, 1.6 Hz), 5.01 (1H, d, J=12.4 Hz), 4.44 (1H, t, J=4.6 Hz), 4.35-4.22 (2H, m), 4.06-3.98 (2H, m), 2.26-2.20 (1H, m), 1.83 (3H, d, J=1.4 Hz), 1.56 (3H, s), 1.46 (3H, s).

### REFERENCES

1. G. M. Blackburn, Gait, M. J., Loakes, D., Williams, D. M., Ed., Nucleic Acids in Chemistry and Biology, (Royal Society of Chemistry, Cambridge, UK, 2006), pp. 503.
2. C. M. Galmarini, J. R. Mackey, C. Dumontet. Nucleoside Analogues and Nucleobases in Cancer Treatment. Lancet Oncol. 3, 415-424 (2002).
3. E. De Clercq. Highlights in Antiviral Drug Research: Antivirals at the Horizon. Med. Res. Rev. 33, 1215-1248 (2013).
4. L. P. Jordheim, D. Durantel, F. Zoulim, C. Dumontet. Advances in the Development of Nucleoside and Nucleotide Analogues for Cancer and Viral Diseases. Nat. Rev. Drug Discov. 12, 447-464 (2013).
5. D. M. Huryn, M. Okabe. AIDS-Driven Nucleoside Chemistry. Chem. Rev. 92, 1745-1768 (1992).
6. J. Shelton et al. Metabolism, Biochemical Actions, and Chemical Synthesis of Anticancer Nucleosides, Nucleotides, and Base Analogs. Chem. Rev. 116, 14379-14455 (2016).
7. B. Ewald, D. Sampath, W. Plunkett. Nucleoside Analogs: Molecular Mechanisms Signaling Cell Death. Oncogene 27, 6522-6537 (2008).
8. K. L. Seley-Radtke, M. K. Yates. The Evolution of Nucleoside Analogue Antivirals: A Review for Chemists and Non-Chemists. Part 1: Early Structural Modifications to the Nucleoside Scaffold. Antiviral Res. 154, 66-86 (2018).
9. M. K. Yates, K. L. Seley-Radtke. The Evolution of Antiviral Nucleoside Analogues: A Review for Chemists and Non-Chemists. Part II: Complex Modifications to the Nucleoside Scaffold. Antiviral Res. 162, 5-21 (2019).
10. H. Ma et al. Characterization of the Metabolic Activation of Hepatitis C Virus Nucleoside Inhibitor Beta-D-2′-Deoxy-2′-Fluoro-2′-C-Methylcytidine (PSI-6130) and Identification of a Novel Active 5′-Triphosphate Species. J. Biol. Chem. 282, 29812-29820 (2007).
11. E. P. Gillis, K. J. Eastman, M. D. Hill, D. J. Donnelly, N. A. Meanwell. Applications of Fluorine in Medicinal Chemistry. J. Med. Chem. 58, 8315-8359 (2015).
12. J. Deval, M. H. Powdrill, C. M. D'Abramo, L. Cellai, M. Gotte. Pyrophosphorolytic Excision of Nonobligate Chain Terminators by Hepatitis C Virus NS5B Polymerase. Antimicrob. Agents Chemother. 51, 2920-2928 (2007).
13. H. Ohrui. 2′-Deoxy-4′-C-Ethynyl-2-Fluoroadenosine, a Nucleoside Reverse Transcriptase Inhibitor, is Highly Potent Against All Human Immunodeficiency Viruses Type 1 and Has Low Toxicity. Chem. Rec. 6, 133-143 (2006).
14. J. T. Witkowski, R. K. Robins, R. W. Sidwell, L. N. Simon. Design, Synthesis, and Broad Spectrum Antiviral Activity of 1-Beta-D-Ribofuranosyl-1,2,4-Triazole-3-Carboxamide and Related Nucleosides. J. Med. Chem. 15,1150-1154 (1972).
15. J. Zeidler, D. Baraniak, T. Ostrowski. Bioactive Nucleoside Analogues Possessing Selected Five-Membered Azaheterocyclic Bases. Eur. J. Med. Chem. 97, 409-418 (2015).
16. G. Ni et al. Review of a-Nucleosides: From Discovery, Synthesis to Properties and Potential Applications. RSC Advances 9, 14302-14320 (2019).
17. G. Gumina, G. Y. Song, C. K. Chu. L-Nucleosides as Chemotherapeutic Agents. FEMS Microbiol. Lett. 202, 9-15 (2001).
18. H. Cui et al. Synthesis and Evaluation of alpha-Thymidine Analogues as Novel Antimalarials. J. Med. Chem. 55,10948-10957 (2012).
19. Chemical Synthesis of Nucleoside Analogues. P. Merino, Ed., (John Wiley & Sons, Inc., 2013), pp. 895.
20. M. Brodszki et al. Synthesis of the Hepatitis B Nucleoside Analogue Lagociclovir Valactate. Org. Process. Res. Dev. 15, 1027-1032 (2011).
21. M. McLaughlin et al. Enantioselective Synthesis of 4′-Ethynyl-2-fluoro-2′-deoxyadenosine (EFdA) via Enzymatic Desymmetrization. Org. Lett. 19, 926-929 (2017).
22. W. T. Markiewicz, M. Wiewi6rowski. A New Type of Silyl Protecting Groups in Nucleoside Chemistry. Nucleic Acids Res. 5, s185-s190 (1978).
23. K. R. Campos et al. The Importance of Synthetic Chemistry in the Pharmaceutical Industry. Science 363, eaat08O5 (2019).
24. M. Peifer, R. Berger, V. W. Shurtleff, J. C. Conrad, D. W. MacMillan. A General and Enantioselective Approach to Pentoses: a Rapid Synthesis of PSI-6130, the Nucleoside Core of Sofosbuvir. J. Am. Chem. Soc. 136, 5900-5903 (2014).
25. M. W. Powner, B. Gerland, J. D. Sutherland. Synthesis of Activated Pyrimidine Ribonucleotides in Prebiotically Plausible Conditions. Nature 459, 239 (2009).
26. J. S. Teichert, F. M. Kruse, O. Trapp. Direct Prebiotic Pathway to DNA Nucleosides. Angew. Chem. Int. Ed. 58, 9944-9947 (2019).
27. D. Chapdelaine et al. A stereoselective approach to nucleosides and 4′-thioanalogues from acyclic precursors. J. Am. Chem. Soc. 131, 17242-17245 (2009).
28. M. Bergeron-Brlek, T. Teoh, R. Britton. A Tandem Organocatalytic alpha-Chlorination-Aldol Reaction that Proceeds with Dynamic Kinetic Resolution: a Powerful Tool for Carbohydrate Synthesis. Org. Lett. 15, 3554-3557 (2013).
29. M. Bergeron-Brlek, M. Meanwell, R. Britton. Direct Synthesis of Imino-C-Nucleoside Analogues and Other Biologically Active Iminosugars. Nat. Commun. 6, 6903 (2015).
30. Meanwell et al., Science, 369, 725-730 (2020) All citations are hereby incorporated by reference.

The present invention has been described with regard to one or more embodiments. However, it will be apparent to persons skilled in the art that a number of variations and modifications can be made without departing from the scope of the invention as defined in the claims. Therefore, although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the specification, the word “comprising” is used as an open-ended term, substantially equivalent to the phrase “including, but not limited to,” and the word “comprises” has a corresponding meaning. It is to be however understood that, where the words “comprising” or “comprises,” or a variation having the same root, are used herein, variation or modification to “consisting” or “consists,” which excludes any element, step, or ingredient not specified, or to “consisting essentially of” or “consists essentially of,” which limits to the specified materials or recited steps together with those that do not materially affect the basic and novel characteristics of the claimed invention, is also contemplated. By “about” is meant a variance (plus or minus) from a value or range of 5% or less, for example, 0.5%, 1%, 1.5%, 2.0%, 2.5%, 3.0%, 3.5%, 4.0%, 4.5%, 5.0%, etc. The elements of the present invention as described may be indicated specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise. Citation of references herein shall not be construed as an admission that such references are prior art to the present invention. All publications are incorporated herein by reference as if each individual publication was specifically and individually indicated to be incorporated by reference herein and as though fully set forth herein. The invention includes all embodiments and variations substantially as hereinbefore described and with reference to the examples.

1. A method of synthesizing a C4′ substituted nucleoside analogue, comprising:
(i) providing a halohydrin ketone compound of the structure: 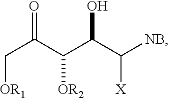
wherein
X is a halogen,
NB is optionally substituted aryl, arylalkyl, heteroaryl, or heteroarylalkyl, and
R₁ and R₂ are each independently H, alkyl, ester, or protecting group, or together form a cyclic group; and
ii) reacting the halohydrin ketone compound with a soft nucleophile to yield a C4′ substituted nucleoside analogue of the structure: 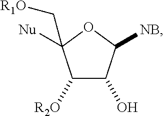
wherein
NB is optionally substituted aryl, arylalkyl, heteroaryl, or heteroarylalkyl,
Nu is a soft nucleophile,
R₁ is H, alkyl, ester, protecting group, or a covalent bond to Nu, and
R₂ is H, alkyl, ester, protecting group, or a covalent bond to Nu.

2. The method of claim 1 wherein R₁ and R₂ are independently —OH, —OC(CH₃)₂O—, —(CH₂)₃—, —CH₂SCH₂—, or —CH₂OCH₂—.

3. The method of claim 1 wherein the halohydrin ketone compound is any one of: 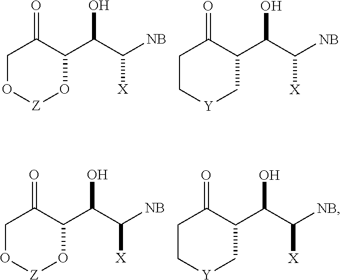
wherein
X is a halogen,
NB is optionally substituted aryl, arylalkyl, heteroaryl, or heteroarylalkyl,
Y, if present, is CH₂, O, S, NR, wherein R is alkyl, aryl, acyl, or carboxylate,
Z, if present, is a protecting group for an alcohol.

4. The method of claim 3 wherein the protecting group for an alcohol is acetonide, silyl protecting group, alkyl protecting group or aryl protecting group.

5. The method of claim 3 wherein the protecting group for an alcohol is tert-butyl(dimethyl)silyl (TBS), triisopropylsilyl (TIPS), trimethylsilyl (TMS), tert-butyldiphenylsilyl (TBDPS), triethylsilyl (TES), 2-(trimethylsilyl)ethoxymethyl ether (SEM), tetrahydropyranyl (THP) ether, acetate (Ac), pivalate (Piv), methoxymethyl or p-methoxybenzyl (PMB).

6. The method of claim 1 wherein the halohydrin ketone compound is either of: 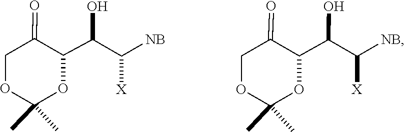
wherein NB is optionally substituted aryl, arylalkyl, heteroaryl, or heteroarylalkyl and X is a halogen.

7. The method of claim 1 wherein the halohydrin ketone compound is either of: 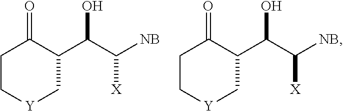
wherein NB is optionally substituted aryl, arylalkyl, heteroaryl, or heteroarylalkyl, X is a halogen, and Y is CH₂, O, S, NR, wherein R is alkyl, aryl, acyl, or carboxylate.

8. The method of claim 1 wherein the halohydrin ketone compound is

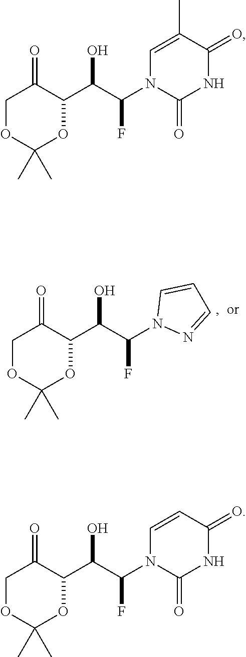

9. The method of claim 1 comprising:
(i) providing a halohydrin ketone compound of the structure: 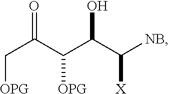
wherein
X is a halogen, and
NB is optionally substituted aryl, arylalkyl, heteroaryl, or heteroarylalkyl, and
PG is a protecting group; and
ii) reacting the halohydrin ketone compound with a soft nucleophile to yield a C4′ substituted nucleoside analogue of the structure: 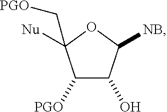
wherein
NB is optionally substituted aryl, arylalkyl, heteroaryl, or heteroarylalkyl,
Nu is a soft nucleophile,
PG is a protecting group that can optionally form a covalent bond to Nu.

10. The method of claim 9 wherein the protecting group is a protecting group for an alcohol.

11. The method of claim 10 wherein the protecting group for an alcohol is acetonide, silyl protecting group, alkyl protecting group or aryl protecting group.

12. The method of claim 10 wherein the protecting group for an alcohol is tert-butyl(dimethyl)silyl (TBS), triisopropylsilyl (TIPS), trimethylsilyl (TMS), tert-butyldiphenylsilyl (TBDPS), triethylsilyl (TES), 2-(trimethylsilyl)ethoxymethyl ether (SEM), tetrahydropyranyl (THP) ether, acetate (Ac), pivalate (Piv), methoxymethyl or p-methoxybenzyl (PMB), including without limitation, acetonide, silyl protecting group, alkyl protecting group or aryl protecting group (including cyclic or acyclic).

13. The method of claim 1 wherein the soft nucleophile is MeCN, N₃⁻, CN⁻, or MeNO₂.

14. The method of claim 1 wherein the halogen is F.

15. The method of claim 1 wherein NB is phenyl, biphenyl, naphthyl, indanyl, indenyl, tetrahydronaphthyl, 2,3-dihydrobenzofuranyl, dihydrobenzopyranyl, 1,4-benzodioxanyl, furan, thiophene, pyrrole, oxazole, thiazole, imidazole, pyrazole, isoxazole, isothiazole, 1,2,3-oxadiazole, triazole, 1,3,4-thiadiazole, tetrazole, pyridine, pyridazine, pyrimidine, 2,6-dichloropyrimidine pyrazine, 1,3,5-triazine, imidazole, benzimidazole, benzoxazole, benzothiazole, indolizine, indole, isoindole, benzofuran, benzothiophene, 1H-indazole, purine, 4H-quinolizine, quinoline, isoquinoline, cinnoline, phthalazine, quinazoline, quinoxaline, 1,8-naphthyridine, pteridine, uracil, thymine, deazadenine, phthalimide, adenine, 5,6-dihydrouracil, 5-methylcytosine, 5-hydroxymethylcytosine, 5,5,5-trifluoromethylthymine, 5-fluorouracil, 2-thiouracil, 4-methylbenzimidazole, hypoxanthine, 7-deazaguanine, or 7-deazaadenine.

16. The method of claim 1 wherein the nucleoside analogue is a locked nucleic acid (LNA), a small molecule therapeutic, or a monomer in an oligonucleotide therapeutic.

17. The method of claim 1 wherein the nucleoside analogue comprises a nitrile moiety can be reduced to an aldehyde.

18. The method of claim 1 wherein the C4′ substituted nucleoside analogue is: 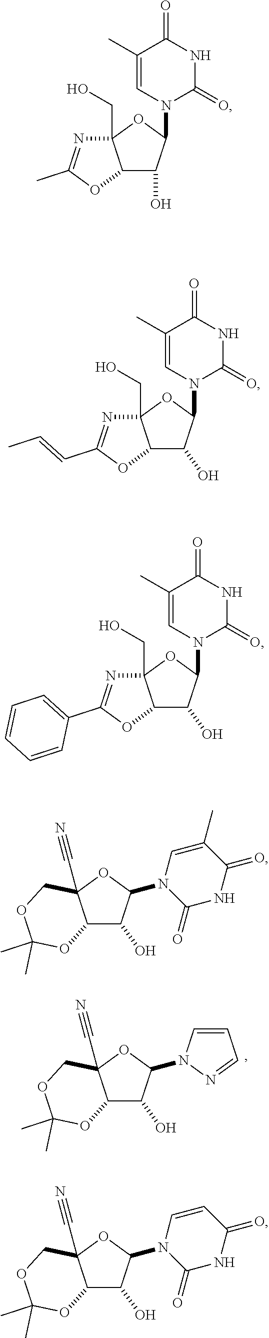 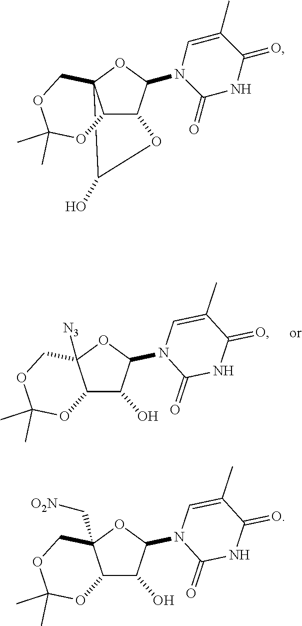 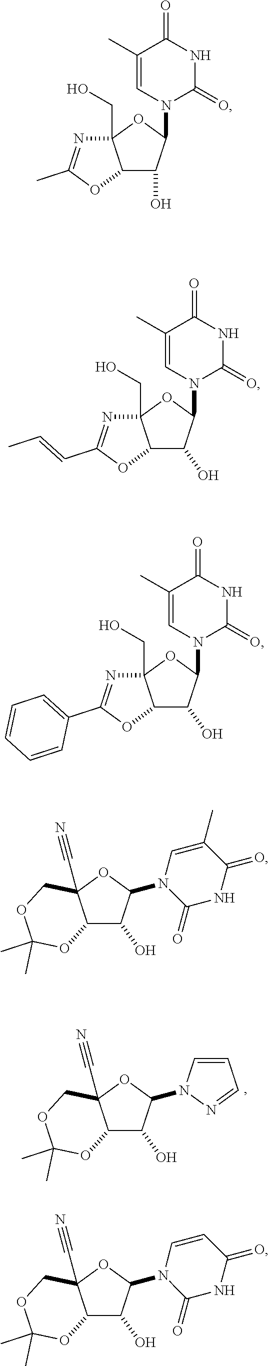 [chemical structure]
